(19) United States
(12) Patent Application Publication
(10) Pub. No.: US 2004/0261495 A1
(43) Pub. Date: Dec. 30, 2004

(54) Method and apparatus for determining the amount of gas contained in a liquid

(76) Inventor: Lawrence B. Kilham, Santa Fe, NM (US)

Correspondence Address: ROBERT W. BECKER & ASSOCIATES, 707 Highway 66 East, Suite B, Tijeras, NM, 87059, US

(21) Appl. No.: 10/608,242

(22) Filed: Jun. 27, 2003

(51) Int. Cl. G01N 33/00; G01N 33/18

(52) U.S. Cl. 73/19.1

(57) ABSTRACT

A method and apparatus for determining the amount of gas contained in a liquid are provided. Air or gas is introduced into a stripping chamber to thereby produce an air or gas atmosphere in the chamber. Liquid in which gas is dissolved is sprayed via a stripping nozzle into the air or gas atmosphere of the stripping chamber to strip gas from the liquid. Air or gas containing gas stripped from the liquid is withdrawn from the stripping chamber. The stripped gas in the withdrawn air or gas is sensed and measured. Liquid remaining in the stripping chamber is withdrawn therefrom.

### BACKGROUND OF THE INVENTION

The present invention relates to a method and apparatus for determining the amount of gas contained in a liquid.

Methods and apparatus are known for measuring dissolved gases in a liquid, for example directly in the liquid itself. A critical problem of such known methods is that most direct in-liquid measurements are not species specific without interference from other types of gases and chemicals. For example, two-electrode ORP (Oxidation-Reduction Potential) or “redox” meters respond to any dissolved chemical or material that changes the ionic potential in the liquid. Other in-liquid measurement methods, such as electrochemical cells, use delicate, permeable membranes to separate the gas from the liquid, with such membranes easily becoming clogged or damaged. Other methods introduce salts, chemicals or other reagents into the liquid, with measurements frequently being based on a color change.

Ultraviolet absorption methods are also known, with the gas concentration then being measured by the amount of UV absorption.

There is a great need for a better way to measure the amount of a gas dissolved or otherwise contained in a liquid, including for the measurement of ozone, volatile organic compounds (VOCs), oxygen, carbon dioxide and other gases. By way of example only, with regard to the measurement of ozone dissolved in, for example, water, ozone has recently become more popular as a strong oxidizing agent in water to disinfect, remove minerals, deodorize, purify, etc. Important applications include sterilizing drinking water, including bottled water, sterilizing food in preparation by washing it with ozonated water, sterilizing packaging and handling equipment in a multitude of industries, etching and washing semiconductor wafers, and making more efficient laundries and car washes. The use of ozone has become even more widely accepted due to recent government approvals of ozone processes, and increasing recognition of the hazards of using traditional sterilization chemicals such as chlorine.

It is therefore an object of the present invention to provide an improved method and apparatus for determining the amount of gas contained in a liquid that overcome the aforementioned drawbacks.

### BRIEF DESCRIPTION OF THE DRAWINGS

This object, and other objects and advantages of the present invention, will appear more clearly from the following specification in conjunction with the accompanying schematic drawings, in which:

FIG. 1 illustrates one exemplary embodiment of the inventive gas stripping and measuring apparatus, and

FIG. 2 is a detailed cross-sectional view of one exemplary embodiment of the stripping chamber of the apparatus of FIG. 1.

### SUMMARY OF THE INVENTION

The method of the present invention for determining the amount of gas contained in a liquid includes the steps of: introducing air or gas into a stripping chamber to thereby produce an air or gas atmosphere in the chamber and to carry stripped gas out of the chamber; spraying liquid in which gas is dissolved into the air or gas atmosphere of the stripping chamber to strip gas from the liquid, withdrawing air or gas containing gas stripped from the liquid from the stripping chamber; sensing and measuring the stripped gas in the withdrawn air or gas; and withdrawing liquid from the stripping chamber.

The key element of the apparatus for practicing the above method is a stripping nozzle in the stripping chamber that receives the liquid in which gas is dissolved; the stripping nozzle then sprays liquid into the air or gas atmosphere of the stripping chamber in order to strip gas from the liquid.

Thus, whereas heretofore known methods generally measure the concentration of a dissolved gas within a solution, with the method and apparatus of the present invention, the gas is removed from the liquid prior to measurement.

Other advantages of the present invention include the fact that the apparatus for practicing the inventive method is very compact. In addition, the inventive process can be used with relatively impure liquids. In addition, the inventive apparatus is very economical to produce. Finally, small flow quantities from a main liquid flow suffice for the inventive method and apparatus.

Further specific features of the present invention will be described in detail subsequently.

### DESCRIPTION OF PREFERRED EMBODIMENTS

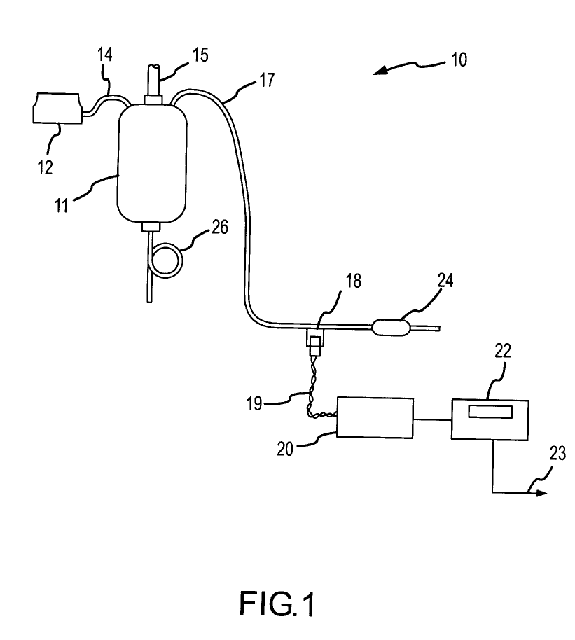

Referring now to the drawings in detail, FIG. 1 shows an overall view of one exemplary embodiment of the inventive gas stripping and measuring apparatus, which is designated generally by the reference numeral 10.

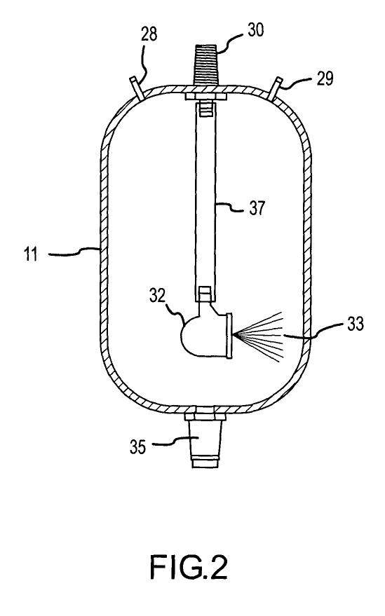

Air or other gas is introduced into a stripping chamber 11, which will be described in greater detail subsequently with reference to FIG. 2. For example, a pump 12 can be used to deliver air or gas to the stripping chamber 11, e.g. via the tubing 14. A generally small proportion of liquid having gas dissolved or otherwise contained therein is branched off from a conduit conveying such liquid for a user's specific application; this conduit can, if desired, contain a pressure gauge upstream from where the small proportion of liquid is branched off. The branched-off liquid stream is delivered, for example via tubing 15, into the stripping chamber 11, for example at the top thereof. As will be explained in connection with FIG. 2, gas is released or stripped from the liquid introduced into the air or gas atmosphere in the stripping chamber 11, and is removed from the stripping chamber by the stream of air or gas that was delivered to the stripping chamber via the pump 12, and which exits the stripping chamber, for example, via tubing 17.

The stream of air or gas containing the gas stripped from the liquid flows through the tubing 17 to gas concentration measurement instruments, such as a sensor 18, where the stripped gas in the stream is sensed. The output from the sensor 18 can be converted into voltage and can be conveyed, for example via the line 19, for further processing, e.g. to the signal conditioner and amplifier 20. The gas concentration can be displayed on a readout, such as on a digital meter 22. Systems controls, which can react to the measured gas concentration, can also be provided at this location or elsewhere, as indicated by the arrow 23.

The stream of air or gas can lead from the sensor 18 to a unit 24 for processing the stripped gas to make the stream safe for discharge. For example, if the stripped gas is ozone, the unit 24 can be an ozone destruct unit, where the ozone is converted back to O₂ and the stream can then safely exit the system into the atmosphere.

Liquid is drained or withdrawn from the stripping chamber 11, for example from the bottom thereof, via the tubing 26. The liquid is preferably drained continuously from the stripping chamber 11, so that the air or gas volume is at least approximately constant in the stripping chamber. The critical feature is that liquid in which gas is dissolved is introduced or sprayed only into the air or gas atmosphere that is present in the stripping chamber 11. In the illustrated embodiment, the tubing 26 is in the form of a P trap in order to create an exit block so that no significant suction is created as the liquid is withdrawn from the stripping chamber 11.

Reference will now be made to the cross-sectional view of the stripping chamber shown in FIG. 2. This stripping chamber provides a novel means for stripping dissolved gas from a liquid. In particular, air or gas, such as an inert gas, which is a good carrier medium that will not interfere with the measurement, enters the stripping chamber 11 via the pump 12 and tubing 14 through the connector 28. The air or gas flows through the stripping chamber 11 and exits the same through the connector 29 to the tubing 17. The liquid in which gas is dissolved is introduced via the tubing 15 and the connector 30 into the air or gas atmosphere of the stripping chamber 11. In particular, the gas-containing liquid is sprayed into the air or gas atmosphere of the stripping chamber 11 via a stripping nozzle 32, thereby stripping gas from the liquid, as a fog is created as indicated by the reference numeral 33, and stripped gas enters the air or gas atmosphere in the stripping chamber 11 and is removed via such air or gas through the connector 29 and tubing 17. The sprayed-out liquid from which gas has been stripped settles at the bottom of the stripping chamber 11 and is removed through the connector 35 and the tubing 26. A section of tubing 37 can be disposed between the connector 30 and the stripping nozzle 32 in order to arrange the stripping nozzle at any desired height within the stripping chamber 11.

As indicated above, when the gas-containing liquid is sprayed into the air or gas atmosphere of the stripping chamber 11 via the stripping nozzle 32, a finely-divided fog is formed. This increases the surface area of the gas-containing liquid, which greatly increases the efficiency of the gas release process.

A specific embodiment of the present invention will now be described in connection with the measurement of ozone dissolved in water. By way of example only, in this embodiment the stripping chamber 11 has a diameter of approximately 50 mm, and a length of 75 mm. The chamber, which must be made of a physically strong, chemically inert, leak proof construction, can be made of schedule 40 PVC. The pump 12, which can be a diaphragm-design pump, delivers air at about 3 liters/minute via the tubing 14 and connector 28 to the stripping chamber 11. Water with ozone dissolved therein is sprayed into the stripping chamber 11 via the stripping nozzle 32. The nozzle 32 can be a stainless steel nozzle having orifices ranging from 0.25 to 1 mm; it operates at pressures of ½ to 2 bar, and flow rates of 0.1 to 0.5 liters/minute. Thus, the stripping nozzle 32 has a relatively low flow rate, and substantially prevents clogging. The stripping nozzle 32 can also be a plastic nozzle.

Ozone is stripped from the water as the ozone-containing water is sprayed by the stripping nozzle 32 into the air atmosphere of the stripping chamber 11, as indicated by the reference numeral 33. The air, which now contains ozone, passes out of the stripping chamber 11 via the connector 29 and the tubing 17, which can, for example, have an inner diameter of 5 mm. After traveling a distance of, for example, approximately half a meter, the ozone-containing air reaches the sensor 18, which can be a heated metal oxide sensor. Such sensors are made, for example, by Eco Sensors, Inc., Santa Fe, N. Mex. Other sensor and measuring equipment could also be used, such as a UV absorption analyzer or an electrochemical cell analyzer. For VOCs, oxygen, carbon dioxide and other non-ozone gases, examples of sensing and measuring equipment could be electrochemical, flame ionization detectors, photo ionization detectors, FTIR, and gas chromatography.

With regard to the ozone-in-water embodiment, the output of the heated metal oxide sensor 18 is converted to voltage for further processing. This is accomplished by amplification and ozone calibration adjustment. An ozone concentration readout can be found at the digital meter 22.

It is to be understood that the concentration of ozone can be calculated on the basis of the amount of ozone that can be released from water (Henry's law) in a given volume. This principle is applicable to any gas dissolved in a liquid.

In this particular embodiment of measuring the amount of ozone dissolved in water, the destruct unit 24 is a catalytic ozone destruct unit, and in particular a manganese oxide ozone destruct unit, which converts the ozone to 02 so that the air can then merely be discharged into the atmosphere.

Although not indicated in the drawings, it should be noted that a pressure regulator can be disposed in the tubing 15 that conveys the liquid in which gas is dissolved to the stripping chamber 11. In the ozone application, since most water lines have a pressure of 14 to 50 psi, the pressure can be set at 10 psi. In addition, a pressure gauge can be located between the pressure regulator and the stripping nozzle in order to indicate if the nozzle has become clogged. In addition, a strainer may be located upstream of the pressure regulator in order to remove any particles from the liquid in which the gas is dissolved.

The present invention is, of course, in no way restricted to the specific disclosure of the specification and drawings, but also encompasses any modifications within the scope of the appended claims.

1. A method for determining the amount of gas contained in a liquid, including the steps of:
providing a stripping chamber;
introducing air or gas into said stripping chamber, thereby producing an air or gas atmosphere therein;
spraying liquid in which gas is dissolved into the air or gas atmosphere of
said stripping chamber to strip gas from said liquid;
withdrawing air or gas containing gas stripped from said liquid from said stripping chamber;
sensing and measuring the stripped gas in said withdrawn air or gas; and
withdrawing liquid from said stripping chamber.

2. A method according to claim 1, wherein said step of introducing air or gas comprises introducing air or gas under pressure into said stripping chamber.

3. A method according to claim 1, wherein said step of withdrawing liquid comprises withdrawing liquid continuously from said stripping chamber.

4. A method according to claim 1, wherein said gas introduced into said stripping chamber is an inert gas.

5. A method according to claim 1, wherein said liquid is water, and said gas contained in said water is ozone.

6. A method according to claim 5, wherein air is introduced into said stripping chamber.

7. A method according to claim 6, wherein said air is introduced into said stripping chamber at a rate of approximately 3 liters per minute.

8. A method according to claim 1, wherein the stripped gas in said withdrawn air or gas is measured by a gas concentration measurement instrument.

9. An apparatus for determining the amount of gas contained in a liquid, comprising:
stripping chamber;
means for introducing air or a gas into said stripping chamber, thereby producing an air or gas atmosphere therein;
a stripping nozzle for receiving liquid in which gas is dissolved and for spraying such liquid into the air or gas atmosphere of said stripping chamber in order to strip gas from said liquid;
means for withdrawing air or gas containing stripped gas from said stripping chamber;
means for sensing and measuring the stripped gas in said withdrawn air or gas; and
means for withdrawing liquid from said stripping chamber.

10. An apparatus according to claim 9, wherein said means for sensing and measuring stripped gas includes a gas concentration measurement instrument.

11. An apparatus according to claim 9, wherein said means for introducing air or gas into said/stripping chamber comprises a pump.

12. An apparatus according to claim 11, wherein said pump is a diaphragm pump.

13. An apparatus according to claim 9, wherein said means for withdrawing air or gas containing stripped gas from said stripping chamber comprises said air or gas introduced into said stripping chamber.

14. An apparatus according to claim 9, wherein said means for withdrawing liquid from said stripping chamber is in the form of a P trap.

15. An apparatus according to claim 9, wherein a stripped gas destruct unit is disposed downstream of said means for sensing and measuring the stripped gas.

16. An apparatus according to claim 9, wherein said stripping nozzle is a stainless steel or plastic nozzle having orifices ranging from 0.25 to 1 mm.

17. A method according to claim 1, which, prior to said spraying step, includes the further step of branching off from a conduit a stream of liquid in which gas is dissolved, wherein said branched-off stream is the liquid subjected to said spraying step.

18. A method according to claim 17, wherein said branched-off stream is not subjected to a pressure increase.

19. A method according to claim 17, which includes the further step of regulating pressure in said branched-off stream down from a pressure in said conduit.

20. An apparatus according to claim 9, which further comprises means for conveying a branch stream, from a conduit of liquid in which gas is dissolved, to said stripping nozzle.

21. An apparatus according to claim 20, wherein no means for increasing pressure is disposed in said branch stream.

22. An apparatus according to claim 20, wherein a pressure regulator is disposed in said branch line.
